(19) United States
(12) Patent Application Publication
(10) Pub. No.: US 2021/0402201 A1
(43) Pub. Date: Dec. 30, 2021

(54) MEDICAL TREATMENT EQUIPMENT, MEDICAL TREATMENT SYSTEM, CONTROL METHOD FOR MEDICAL TREATMENT EQUIPMENT, AND NON-TRANSITORY STORAGE MEDIUM STORING CONTROL PROGRAM FOR MEDICAL TREATMENT EQUIPMENT

(71) Applicant: OMRON HEALTHCARE Co., Ltd., Kyoto (JP)

(72) Inventors: Mitsuru SAMEJIMA, Kyoto (JP); Tamaki ITO, Kyoto (JP); Keisuke YAMADA, Kyoto (JP)

(21) Appl. No.: 17/447,248

(22) Filed: Sep. 9, 2021

Related U.S. Application Data
(63) Continuation of application No. PCT/JP2020/006646, filed on Feb. 19, 2020.

(30) Foreign Application Priority Data
Mar. 13, 2019 (JP) 2019-045928

(51) Int. Cl. A61N 5/06; A61B 5/026; A61B 5/00; A61B 5/107; A61N 5/02

(52) U.S. Cl. CPC A61N 5/06; A61B 5/026; A61B 5/4836; A61N 7/00; A61B 5/6828; A61N 5/02; A61B 5/1071

(57) ABSTRACT

To provide medical treatment equipment, a medical treatment system, a control method for the medical treatment equipment, and a non-transitory storage medium storing a control program for the medical treatment equipment that can perform an optimal treatment in accordance with a state of the affected area. Medical treatment equipment acquires measurement information from a bending sensor of a sensor unit attached to the knee region of a user, evaluates a movable range of the knee region based on the measurement information (step S2), and controls the output of each of a first electrode pad and a second electrode pad based on the evaluation result (step S3, step S4).

### CROSS-REFERENCE TO RELATED APPLICATIONS

This application is the U.S. national stage application filed pursuant to 35 U.S.C. 365(c) and 120 as a continuation of International Patent Application No. PCT/JP2020/006646, filed Feb. 19, 2020, which application claims priority to Japanese Patent Application No. 2019-045928, filed Mar. 13, 2019, which applications are incorporated herein by reference in their entireties.

### TECHNICAL FIELD

The present invention relates to medical treatment equipment, a medical treatment system, a control method for medical treatment equipment, and a non-transitory storage medium storing a control program for medical treatment equipment.

### BACKGROUND ART

For knee pain, lumbar pain, shoulder stiffness, sprain, muscle fatigue, muscle pain, or the like, medical treatment equipment, such as a low-frequency treatment device, an infrared treatment device, a microwave treatment device, or an ultrasonic treatment device, which utilizes electrically generated energy (current, electromagnetic wave, ultrasound) to improve the affected area, is used. For example, Patent Document 1 describes an electrical stimulation device including a trunk portion electrode attachment member, a left knee electrode attachment member, and a right knee electrode attachment member.

### CITATION LIST

### Patent Literature

Patent Document 1: WO 2013/124911

### SUMMARY OF INVENTION

### Technical Problem

In the medical treatment equipment described above, a patient or medical professional using the medical treatment equipment sets an operating state of the equipment such as low frequency output setting, electromagnetic wave output setting, or ultrasound output setting, and then the affected area is treated by the medical treatment equipment operating in the operating state. Such an operating state should be optimally set in accordance with a state of the affected area such as how much the affected area moves or how much the blood flow is disrupted in the affected area. Since the patient's condition is determined based on patient's subjectivity or medical professional's experience, the condition may not be accurate; therefore, an efficient treatment may not be performed. Such a problem is not recognized in Patent Document 1.

An object of the present invention is to provide medical treatment equipment, a medical treatment system, a control method for medical treatment equipment, and a control program for medical treatment equipment that can perform an optimal treatment in accordance with a state of the affected area.

### Solution to Problem

(1) Medical treatment equipment for treating a body of a user with energy generated by energization, the medical treatment equipment including:
a measurement information acquisition unit configured to acquire, from a blood flow sensor attached to a portion of a surface of the body of the user to measure a blood flow, measurement information of the blood flow;
a body state evaluation unit configured to evaluate a blood circulation state of the portion of the body based on the measurement information; and
an operating state control unit configured to control an operating state of a generation source of the energy, based on an evaluation result from the body state evaluation unit.

According to (1), the blood flow sensor is worn on the affected area to be treated by the medical treatment equipment, and thus a blood circulation state of the portion of the body of the user can be evaluated based on the measurement information acquired from the blood flow sensor. An operating state of the generation source of the energy applying energy to the affected area (for example, the magnitude of the energy or the location in which the energy is generated) can be optimized based on the evaluation result. Therefore, even a person without experience or knowledge can efficiently treat the affected area of the user. Additionally, the blood circulation state of the affected area of the user can be evaluated based on the measurement information from the blood flow sensor; therefore, a state of the energy applied to the affected area can be optimized and an efficient treatment can be provided according to the blood circulation state.

(2) The medical treatment equipment described in (1), wherein the sensor includes a motion sensor for measuring an amount of movement, and the body state evaluation unit evaluates a movable range of the portion of the body, based on measurement information of the motion sensor.

According to (2), a movable range of, for example, the joint of the user can be evaluated based on the measurement information of the motion sensor. Therefore, in accordance with a state of movement of the joint, a state of the energy applied to the affected area can be optimized and an efficient treatment can be provided.

(3) The medical treatment equipment described in (2), wherein in a case where an evaluation value of the movable range is at a threshold or lower, the operating state control unit increases energy generated from the generation source of the energy, compared with a case where the evaluation value of the movable range exceeds the threshold.

According to (3), treatment is performed with large energy when a movable range is narrow. Therefore, expansion of the movable range can be expected, which can lead to symptom improvement of the affected area.

(4) The medical treatment equipment described in (2) or (3), wherein the motion sensor includes at least one of a bending sensor configured to measure an amount of bending of the portion to which the sensor is attached, an acceleration sensor, and an angular velocity sensor.

According to (4), a movable range of the affected area can be evaluated with high accuracy.

(5) The medical treatment equipment described in (1), wherein the sensor includes a blood flow sensor for measuring a blood flow, and
the body state evaluation unit evaluates a blood circulation state of the portion of the body, based on measurement information of the blood flow sensor.

According to (5), a blood circulation state of the affected area of the user can be evaluated based on the measurement information of the blood flow sensor. Therefore, according to the blood circulation state, a state of the energy applied to the affected area can be optimized and an efficient treatment can be provided.

(6) The medical treatment equipment described in (5), wherein in a case where an evaluation value of the blood circulation state is at a threshold or lower, the operating state control unit increases energy generated from the generation source of the energy, compared with a case where the evaluation value of the blood circulation state exceeds the threshold.

According to (6), treatment is performed with large energy when a blood circulation state is poor. Therefore, improvement in the blood circulation state can be expected, which can lead to symptom improvement of the affected area.

(7) The medical treatment equipment described in (5), wherein the sensor includes a plurality of the blood flow sensors,
the generation source includes electrode pads corresponding to sites of the body of the user to which the plurality of the blood flow sensors are respectively attached, and
the operating state control unit controls an amount of energy generated from each of the electrode pads, based on an evaluation result of the blood circulation state.

According to (7), in a case where, for example, a location in which a blood circulation state is poor and a location in which a blood circulation state is good are mixed in the affected area, the location in which the blood circulation state is poor can be treated with large energy applied. Therefore, improvement in the blood circulation state can be expected, which can lead to symptom improvement of the affected area.

(8) The medical treatment equipment described in (7), wherein the body state evaluation unit evaluates a blood circulation state for each of the sites to which the blood flow sensors are respectively attached, based on measurement information of each of the blood flow sensors, and
the operating state control unit increases an amount of energy generated from the electrode pad corresponding to the site in which an evaluation value of the blood circulation state is at a threshold or lower, to be greater than an amount of energy generated from the electrode pad corresponding to the site in which an evaluation value of the blood circulation state exceeds the threshold.

According to (8), treatment is performed with large energy when a blood circulation state is poor. Therefore, improvement in the blood circulation state can be expected, which can lead to symptom improvement of the affected area.

(9) A medical treatment system includes the medical treatment equipment described in any one of (1) to (8) and the sensor.

(10) A control method for medical treatment equipment for treating a body of a user with energy generated by energization, the control method including: a measurement information acquisition step of acquiring, from a blood flow sensor attached to a portion of the body of the user to measure a blood flow, measurement information of the blood flow;
a body state evaluation step of evaluating a blood circulation state of the portion of the body based on the measurement information; and
an operating state control step of controlling an operating state of a generation source of the energy, based on an evaluation result from the body state evaluation step.

(11) A non-transitory storage medium storing a control program for medical treatment equipment for treating a body of a user with energy generated by energization, the non-transitory storage medium storing the control program for the medical treatment equipment, allowing a computer to execute:
a measurement information acquisition step of acquiring, from a blood flow sensor attached to a portion of the body of the user to measure a blood flow, measurement information of the blood flow;
a body state evaluation step of evaluating a blood circulation state of the portion of the body based on the measurement information; and
an operating state control step of controlling an operating state of a generation source of the energy, based on an evaluation result from the body state evaluation step.

### Advantageous Effects of Invention

According to an embodiment of the present invention, medical treatment equipment, a medical treatment system, a control method for the medical treatment equipment, and a non-transitory storage medium storing a control program for the medical treatment equipment that can perform an optimal treatment in accordance with a state of the affected area can be provided.

### BRIEF DESCRIPTION OF DRAWINGS

FIG. 1 is a schematic diagram illustrating an overall configuration of a medical treatment system 100.

FIG. 2 is an exploded perspective view illustrating an overall configuration of medical treatment equipment 1 illustrated in FIG. 1.

FIG. 3 is a diagram illustrating a wrapped state where the medical treatment equipment 1 illustrated in FIG. 2 is wrapped around a region below the right knee.

FIG. 4 is a diagram illustrating positions in which a first electrode pad 41 and a second electrode pad 42 are in contact with the body surface in the wrapped state illustrated in FIG. 3.

FIG. 5 is a schematic diagram illustrating an overall configuration of a sensor unit 3 illustrated in FIG. 1.

FIG. 6 is a diagram illustrating a functional block of a control unit 50 of the medical treatment equipment 1 illustrated in FIG. 2.

FIG. 7 is a schematic diagram for illustrating movements in a pre-measurement.

FIG. 8 is a flowchart for illustrating the operation of the medical treatment equipment 1 illustrated in FIG. 1 during treatment.

FIG. 9 is a diagram illustrating an overall configuration of medical treatment equipment 200 according to another embodiment of the present invention.

FIG. 10 is a flowchart for illustrating the operation of the medical treatment equipment 200 illustrated in FIG. 9 during treatment.

### DESCRIPTION OF EMBODIMENTS

Overview of Medical Treatment Equipment of Embodiment

Medical treatment equipment according to an embodiment of the present invention serves to treat the body of a user with energy such as current, ultrasound, or electromagnetic wave (microwave or infrared rays) generated by energization. The medical treatment equipment acquires, from a sensor for measuring the physical quantity (for example, the amount of movement or blood flow of the portion of the body) of a portion (for example, the knee or hip) of the body of the user to which the sensor is attached, measurement information of the physical quantity and, based on the measurement information, evaluates a state of the portion of the body of the user to which the sensor is attached (for example, a movable range or blood circulation state). Then, based on the evaluation results of the state of the portion of the body of the user, the medical treatment equipment controls an operating state (for example, the magnitude of output) of a generation source of the aforementioned energy (for example, an electrode pad, an ultrasonic probe, or an electromagnetic wave irradiation device).

Accordingly, even in a user who does not have knowledge or experience, the generation source of energy is operated in an operating state matching a state of the affected area of the user; therefore, an optimal treatment can be performed for each patient.

Specific Configuration of Medical Treatment System Including Medical Treatment Equipment of Embodiment

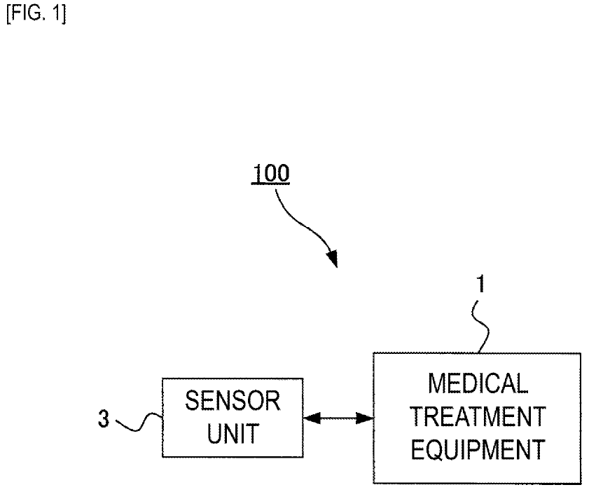

FIG. 1 is a schematic diagram illustrating an overall configuration of a medical treatment system 100. The medical treatment system 100 illustrated in FIG. 1 includes medical treatment equipment 1 and a sensor unit 3. The medical treatment equipment 1 and the sensor unit 3 are configured so as to communicate, for example, by wireless communication. Note that the medical treatment equipment 1 and the sensor unit 3 may be integrally formed.

The medical treatment equipment 1 is equipment for treating the body of a user with energy generated by energization. Here, “energy” refers to current, ultrasound, electromagnetic wave (infrared rays or microwave), or the like. The medical treatment equipment 1 includes a generation source of energy, and the generation source of energy is: an electrode pad directly affixed to the body of the user to apply current to the body, an ultrasonic probe for applying ultrasound waves to the body of the user, an electromagnetic wave irradiation device for irradiating electromagnetic waves to the body of the user, or the like.

In the following example, a low-frequency treatment device configured to perform treatment by applying a low frequency pulse current of, for example, 1 Hz or higher and 1200 Hz or lower to the body of the user will be described as an example of the medical treatment equipment 1.

Configuration in Details of Medical Treatment Equipment

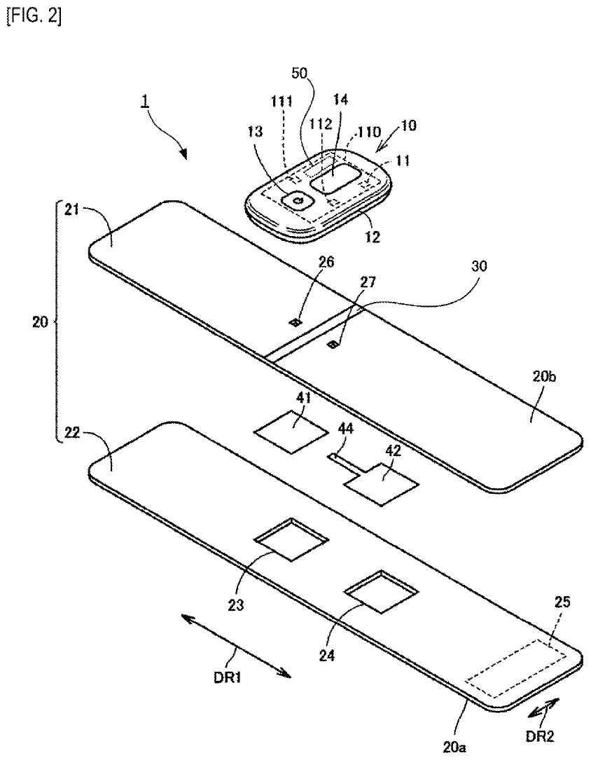

FIG. 2 is an exploded perspective view illustrating an overall configuration of the medical treatment equipment 1 illustrated in FIG. 1. As illustrated in FIG. 2, the medical treatment equipment 1 includes a main body portion 10, a holding member 20, a first electrode pad 41, and a second electrode pad 42.

The medical treatment equipment 1 is a low-frequency treatment device configured to relieve pain by applying the aforementioned low-frequency pulse current to the first electrode pad 41 and the second electrode pad 42 that are in contact with the body surface of the user.

The holding member 20 is configured, for example, so as to be wrapped around the right lower limb of the user. The holding member 20 has an elongated shape in a state before being wrapped. The holding member 20 has a substantially rectangular shape in a planar view.

The holding member 20 holds the first electrode pad 41 and the second electrode pad 42, and maintains a state of the first electrode pad 41 and the second electrode pad 42 pressed against the body surface of the user.

In a wrapped state of being wrapped around the right lower limb, the holding member 20 has a length direction (direction DR1) corresponding to a circumferential direction and a width direction (direction DR2) orthogonal to the length direction. The holding member 20 includes: a first main surface 20a facing the body surface of the user in the wrapped state; and a second main surface 20b positioned on an opposite side of the first main surface 20a.

The holding member 20 includes a first member 21, a second member 22, and a mark portion 30. The first member 21 and the second member 22 have flexibility. The first member 21 is a member forming an outer circumferential side of the holding member 20 in the wrapped state. A surface of the first member 21, which corresponds to the outer circumferential side in the wrapped state (the second main surface 20b of the holding member 20) is configured to lock a surface fastener 25 described below. The first member 21 is provided with opening portions 26, 27 at positions corresponding to a first terminal 111 and a second terminal 112 of the main body portion 10 described below.

The second member 22 is a member forming an inner circumferential side of the holding member 20 in the wrapped state. A surface of the second member 22, which corresponds to the inner circumferential side in the wrapped state (the first main surface 20a of the holding member 20) is provided with the surface fastener 25. In the wrapped state, the surface fastener 25 is locked to the second main surface 20b, and thereby the holding member 20 is fixed to the right lower limb.

The second member 22 is provided with window portions 23, 24 at positions corresponding to the first electrode pad 41 and the second electrode pad 42. The window portions 23, 24 are provided, and thereby the first electrode pad 41 and the second electrode pad 42 can be brought into contact with the body surface of the user in the wrapped state.

The main body portion 10 is fixed to the holding member 20. The main body portion 10 is located on the second main surface 20b of the holding member 20. The main body portion 10 includes a current supply portion 11 and a case 12. The current supply portion 11 supplies current to the first electrode pad 41 and the second electrode pad 42. The current supply portion 11 is mounted on a circuit board 110.

The circuit board 110 is provided with the first terminal 111 and the second terminal 112. The first terminal 111 is electrically connected to the first electrode pad 41. The second terminal 112 is electrically connected by a wire portion 44 to the second electrode pad 42. Note that the first electrode pad 41 may be electrically connected to the first terminal 111 by wiring, without being directly connected to the first terminal 111.

The circuit board 110 is further provided with a control unit 50. The control unit 50 includes a processor such as a central processing unit (CPU), a random access memory (RAM), a read only memory (ROM), and the like, and controls operation of each portion of the medical treatment equipment 1 in accordance with a program. Programs including a control program are stored in the ROM of the control unit 50.

The case 12 has a box shape. The case 12 accommodates therein the current supply portion 11 and the circuit board 110. The case 12 is disposed on the second main surface 20b of the holding member 20. A power button 13 and a button 14 for performing other various operations are disposed on the front side of the case 12.

The mark portion 30 is an indication for guiding the first electrode pad 41 and the second electrode pad 42 to a dermatome in which pain can be relieved, by aligning the first electrode pad 41 and the second electrode pad 42 with specific positions (reference positions) on the right lower limb when pressing the first electrode pad 41 and the second electrode pad 42 against the body surface.

The mark portion 30 has a linear shape. The mark portion 30 extends along the width direction of the holding member 20. The mark portion 30 is disposed on the second main surface 20b of the holding member 20.

The mark portion 30 is disposed to be exposed from the main body portion 10. Accordingly, when the user is wrapping the holding member 20, the user can easily recognize the mark portion 30. Therefore, the mark portion 30 can be easily aligned with a specific position of the user as described below.

The first electrode pad 41 and the second electrode pad 42 have a polygonal shape. Note that the polygonal shape also includes shapes with rounded corners. The first electrode pad 41 and the second electrode pad 42 have, for example, a square shape. Note that the first electrode pad 41 and the second electrode pad 42 are not limited to a polygonal shape, and the first electrode pad 41 and the second electrode pad 42 may have an oval shape such as an elliptical shape, a long circular shape, and a racetrack shape; or a circular shape.

The first electrode pad 41 and the second electrode pad 42 are fixed to the holding member 20. The first electrode pad 41 and the second electrode pad 42 are disposed side by side in the aforementioned length direction. The first electrode pad 41 is disposed on one side in the aforementioned length direction with respect to the mark portion 30. The second electrode pad 42 is disposed on the other side in the aforementioned length direction with respect to the mark portion 30.

The first electrode pad 41 and the second electrode pad 42 are exposed through the window portions 23, 24 of the second member 22. Therefore, the first electrode pad 41 and the second electrode pad 42 can be brought into contact with the body surface of the user in the wrapped state.

The first electrode pad 41 and the second electrode pad 42 are electrode pads for applying a current to the user. With the first electrode pad 41 and the second electrode pad 42 in contact with the body surface of the user, a current is supplied to the first electrode pad 41 and the second electrode pad 42, and thus the current can be applied to the user.

Wearing Mode of Medical Treatment Equipment

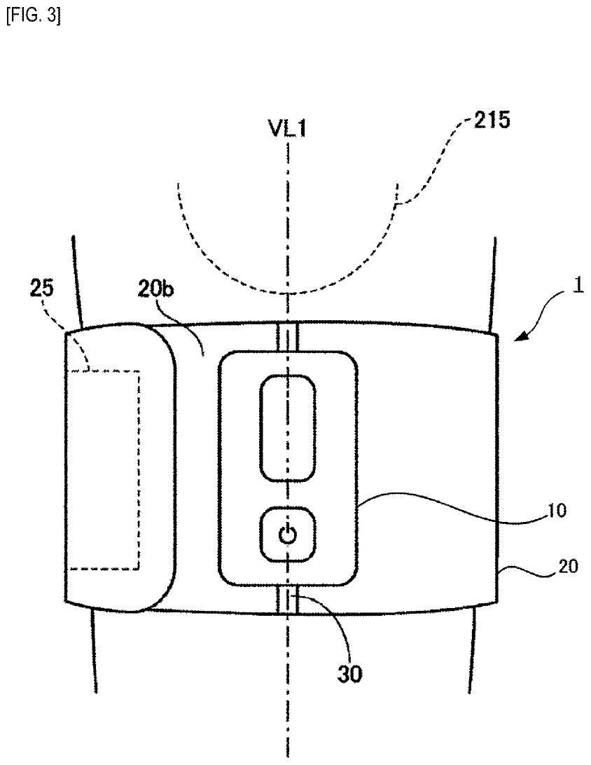

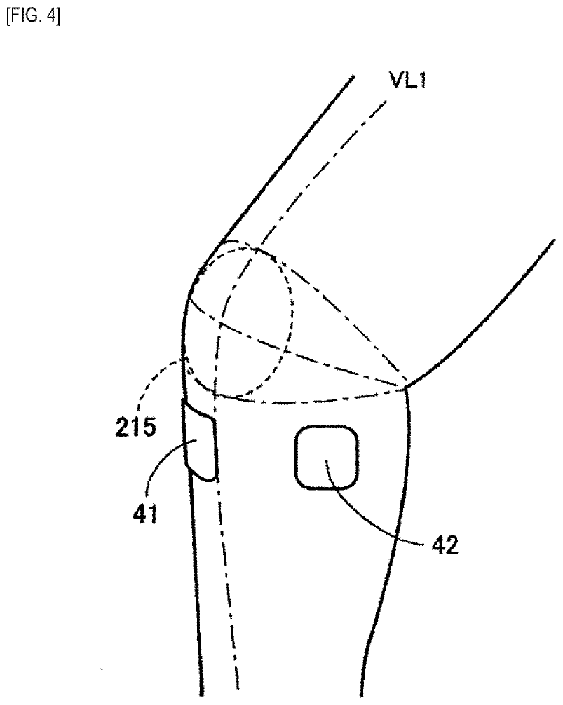

FIG. 3 is a diagram a wrapped state where the medical treatment equipment 1 illustrated in FIG. 2 is wrapped around a region below the right knee. FIG. 4 is a diagram illustrating positions in which the first electrode pad 41 and the second electrode pad 42 are in contact with the body surface in the wrapped state illustrated in FIG. 3. The wrapped state where the medical treatment equipment 1 is wrapped around the region below the right knee will be described with reference to FIGS. 3 and 4.

In the event of wrapping the medical treatment equipment 1 around the region below the right knee as illustrated in FIG. 3, the holding member 20 is wrapped such that the mark portion 30 overlaps with an imaginary line VL1 passing through the center of a right kneecap 215 in a length direction of the right lower limb as viewed from the front of the user. The center of the right kneecap 215 is a reference position for the living body. Note that it is preferable to overlap the imaginary line VL1 with the center line of the mark portion 30. With the imaginary line VL1 and the mark portion 30 overlapped, the surface fastener 25 is locked to the second main surface 20b. The mark portion 30 is positioned so as to overlap with the aforementioned imaginary line VL1, and thus the first electrode pad 41 and the second electrode pad 42 are respectively guided to target positions.

Configuration in details of Sensor Unit

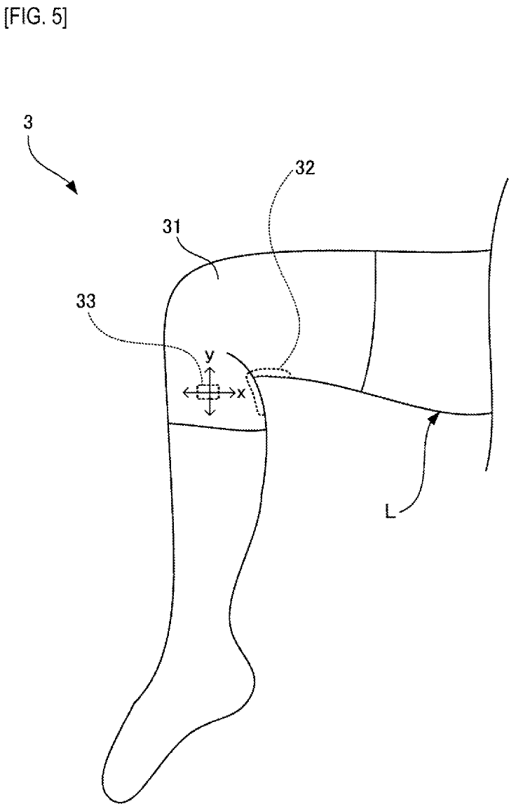

FIG. 5 is a schematic diagram illustrating an overall configuration of the sensor unit 3 illustrated in FIG. 1. The sensor unit 3 is used when worn on the knee region of the user. FIG. 5 schematically illustrates a state where the sensor unit 3 is worn on the knee region of a right lower limb L of the user.

As illustrated in FIG. 5, the sensor unit 3 includes: a cylindrical supporter section 31 having a hollow portion through which the lower limb L is passed; and a bending sensor 32 and an acceleration sensor 33 that are housed in the supporter section 31.

The bending sensor 32 is a sensor having flexibility and configured to measure the amount of bending of a portion to which the bending sensor is attached. For example, a sensor configured such that a resistance value increases in accordance with the amount of bending is used as the bending sensor 32, and a voltage value determined by the resistance value is measured as information of the amount of bending. The bending sensor 32 is disposed at a position opposite the back side of the knee region of the user in the wearing state of the sensor unit 3 illustrated in FIG. 5. The measurement information measured by the bending sensor 32 is transmitted via a communication interface (not illustrated) to the medical treatment equipment 1.

The acceleration sensor 33 is arranged below the knee region of the user in the wearing state of the sensor unit 3 illustrated in FIG. 5, and measures movement of a portion below the knee region (the calf) of the user on which the sensor unit 3 is worn. A sensor configured to measure, for example, acceleration in the x-axis direction and acceleration in the y-axis direction is used as the acceleration sensor 33. The measurement information measured by the acceleration sensor 33 is transmitted via a communication interface (not illustrated) to the medical treatment equipment 1.

The bending sensor 32 and the acceleration sensor 33 respectively constitute sensors for measuring the physical quantity.

Function of Control Unit

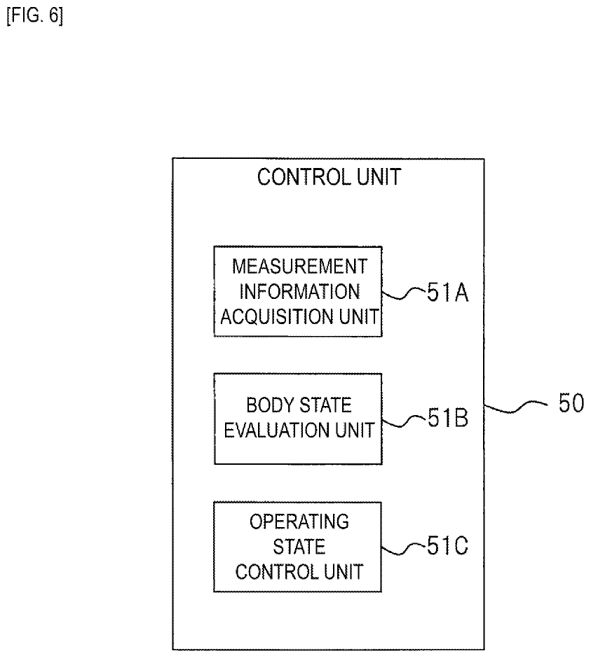

FIG. 6 is a diagram illustrating a functional block of the control unit 50 of the medical treatment equipment 1 illustrated in FIG. 2. The CPU of the control unit 50 executes the aforementioned control program stored in the ROM and thereby functions as a measurement information acquisition unit 51A, a body state evaluation unit 51B, and an operating state control unit 51C.

The measurement information acquisition unit 51A acquires measurement information measured by the bending sensor 32 and the acceleration sensor 33 of the sensor unit 3 from the sensor unit 3. In the medical treatment system 100, it is assumed that the sensor unit 3 is used to perform a pre-measurement to measure a state of the calf of the user, before starting treatment of the affected area of the user with the medical treatment equipment 1. The measurement information acquisition unit 51A acquires the measurement information measured by the bending sensor 32 and the acceleration sensor 33 in the pre-measurement from the sensor unit 3.

The body state evaluation unit 51B evaluates, based on the measurement information acquired by the measurement information acquisition unit 51A, a movable range of a portion (the calf) of the body of the user on which the sensor unit 3 is worn.

Concrete Examples of Pre-Measurement

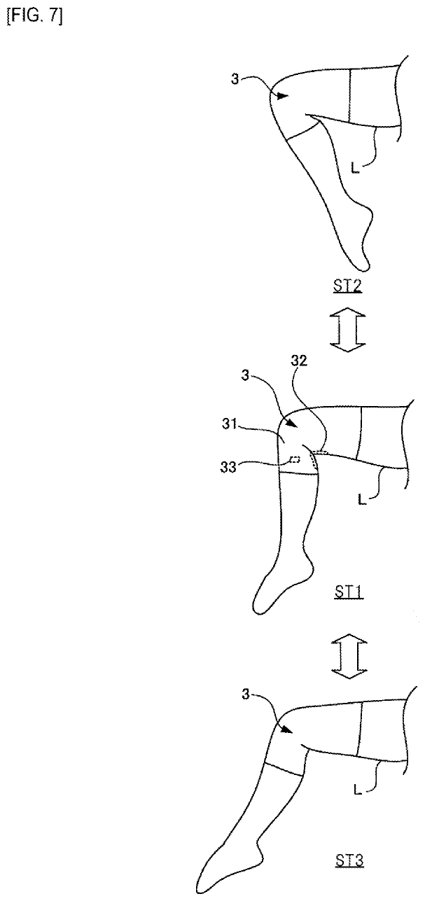

FIG. 7 is a schematic diagram for illustrating describing movements in the pre-measurement. In FIG. 7, a state ST1 where the lower limb L of the user wearing the sensor unit 3 on the knee region is at an angle of approximately 90 degrees formed by a line connecting the knee region and the thigh and a line connecting the knee region and the ankle; a state ST2 where with respect to the state ST1, the calf of the lower limb L is bent toward the body trunk of the user; and a state ST3 where with respect to the state ST1, the calf of the lower limb L is bent toward the opposite side of the body trunk of the user are illustrated.

In the pre-measurement, first, the lower limb L of the user is held in the state ST1, and then the calf is moved and changed to the state ST2. Thereafter, the lower limb L of the user is returned from the state ST2 to the state ST1 and is subsequently changed to the state ST3. This series of movements is performed by the user. When moving the calf from the state ST1 to the state ST2, the user moves the calf to a position until or immediately before pain occurs in the knee region. Likewise, when moving the calf from the state ST1 to the state ST3, the user moves the calf to a position until or immediately before pain occurs in the knee region. The operation of the control unit 50 during the pre-measurement will be described below.

First, the user presses down the button 14 while holding the lower limb L in the state ST1. When the button 14 is pressed down, the measurement information acquisition unit 51A starts acquiring measurement information. When the measurement information is acquired, the body state evaluation unit 51B sets the measurement information of the bending sensor 32 and the acceleration sensor 33 in the state ST1 as a reference value.

Next, the user moves the calf toward the body trunk. The body state evaluation unit 51B calculates the amount of change from the aforementioned reference value of the measurement information generated in accordance with this movement, and determines a bending angle of the calf with respect to the state ST1 from the amount of change at the time when the amount of change is maximum.

Next, the user moves the calf toward the opposite side of the body trunk. The body state evaluation unit 51B calculates the amount of change from the aforementioned reference value of the measurement information generated in accordance with this movement, and determines a bending angle of the calf with respect to the state ST1 from the amount of change at the time when the amount of change is maximum.

The body state evaluation unit 51B thus determines the bending angle at the time when the calf is moved toward the body trunk and the bending angle when the calf is moved toward the opposite side of the body trunk, and determines a value of the sum of absolute values of these bending angles as a movable range of the calf of the user.

Note that since the movable range can be determined independently by each of the measurement information of the bending sensor 32 and the measurement information of the acceleration sensor 33, only one of the bending sensor 32 and the acceleration sensor 33 may be included in the sensor unit 3. However, for example, the bending sensor 32 and the acceleration sensor 33 are disposed in the sensor unit 3, and thereby an average of a movable range determined based on the measurement information of the bending sensor 32 and a movable range determined based on the measurement information of the acceleration sensor 33 can be used as a final movable range, and determination accuracy on the movable range can be increased. In addition, the movable range can be determined by using an angular velocity sensor instead of the acceleration sensor 33, or further by combining the angular velocity sensor with the acceleration sensor 33. In other words, the sensor unit 3 may be equipped with one or both of: the bending sensor 32; and a motion sensor that includes at least one of the acceleration sensor 33 and the angular velocity sensor.

The body state evaluation unit 51B generates a numerical index (an evaluation value) for evaluating the movable range, in accordance with the size of the determined movable range of the calf. The wider the movable range is, the greater the evaluation value is. For example, information of a movable range of a healthy person is held in advance as a reference movable range, and a determined movable range is narrower than the reference movable range. In such a case, the evaluation value is set to a low value (for example, “1”). In a case where a determined movable range is the reference movable range or wider, the evaluation value is set to a high value (for example, “2”). The evaluation value is not limited to two levels, and may be divided into three or more levels.

The operating state control unit 51C illustrated in FIG. 6 controls an operating state of a generation source of energy (here, the first electrode pad 41 and the second electrode pad 42) applied to the affected area, based on the evaluation results (specifically, the evaluation value described above) from the body state evaluation unit 51B.

Operation During Treatment

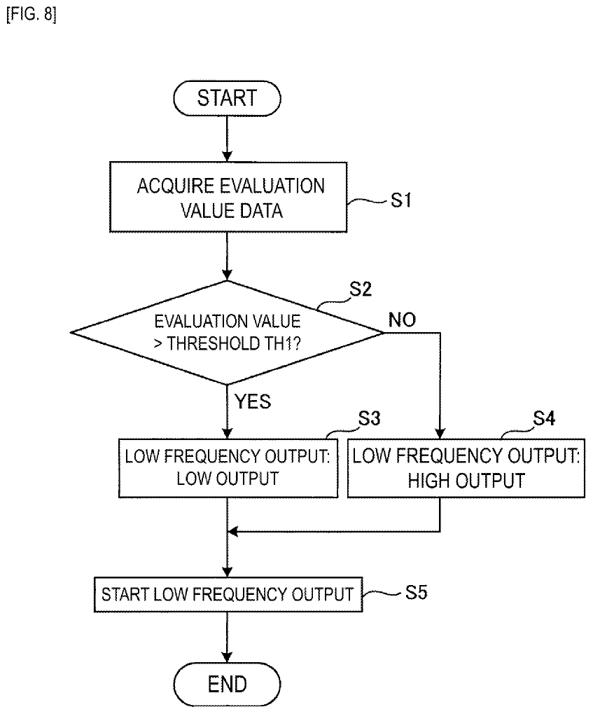

FIG. 8 is a flowchart for illustrating the operation of the medical treatment equipment 1 illustrated in FIG. 1 during treatment. When an instruction to start a low frequency treatment is made by operation of the button 14, the operating state control unit 51C acquires data of the evaluation value of the movable range of the user stored in the RAM in the pre-measurement (step S1). Then, the operating state control unit 51C determines whether the evaluation value in the acquired data exceeds a threshold TH1 (step S2). The threshold TH1 is set to, for example, “1”.

When the evaluation value exceeds the threshold TH1 (step S2: YES), the operating state control unit 51C sets the output (an amplitude value) of a low frequency current supplied to the first electrode pad 41 and the second electrode pad 42 to a predetermined value (step S3). When the evaluation value is at the threshold TH1 or smaller (step S2: NO), the operating state control unit 51C sets the output of the low frequency current supplied to the first electrode pad 41 and the second electrode pad 42 to a value larger than the aforementioned predetermined value (step S4).

After step S3 or step S4, the low frequency current of the amplitude value set in step S3 or step S4 is supplied to the first electrode pad 41 and the second electrode pad 42 (step S5), and a treatment of a region below the knee of the user is started.

Effect of Medical Treatment System of Embodiment

As described above, according to the medical treatment system 100, the sensor unit 3 is worn on the affected area to be treated by the medical treatment equipment 1, and thus a state of the affected area of the body of the user can be evaluated based on measurement information acquired from the sensor unit 3. The size of the low frequency current applied to the affected area can be optimized based on the evaluation results. Therefore, even a person with no experience or knowledge can efficiently treat the affected area by using the medical treatment equipment 1.

Note that in the example described above, a portion at or near the knee region is a treatment target portion of the medical treatment equipment 1; however, the treatment target portion is not limited thereto. For example, the medical treatment equipment 1 may be worn and utilized on the upper arm, the forearm, the shoulder, or the waist. In that case, the sensor unit 3 may be configured to be worn on the elbow, the shoulder, or the waist to transmit information of movement of the elbow, the shoulder, or the waist to the medical treatment equipment 1 in the pre-measurement, and the control unit 50 may be configured to evaluate a movable range of the elbow, the shoulder, or the waist based on the information of this movement.

Medical Treatment Equipment of Another Embodiment

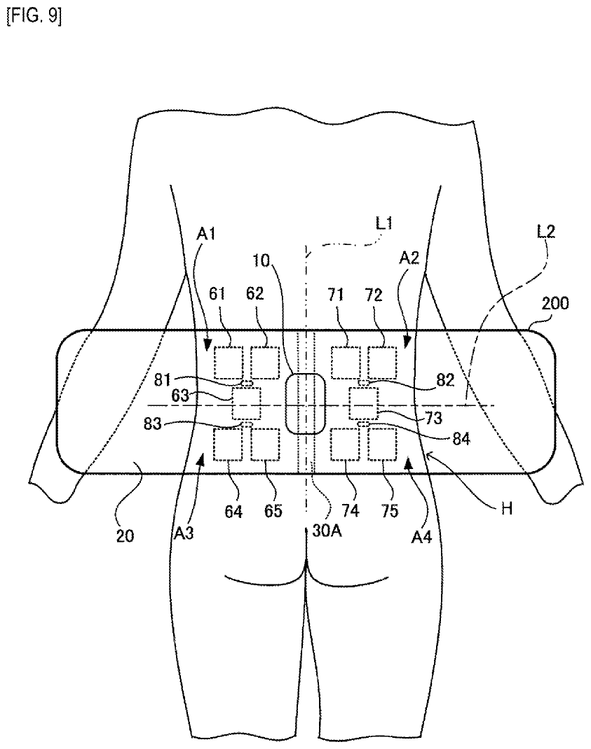

FIG. 9 is a diagram illustrating an overall configuration of medical treatment equipment 200 according to another embodiment of the present invention. The medical treatment equipment 200 is configured in the same manner as the medical treatment equipment 1 except that in the medical treatment equipment 1 illustrated in FIG. 2, the first electrode pad 41 and the second electrode pad 42 are changed to electrode pads 61 to 65 and electrode pads 71 to 75, blood flow sensors 81, 82, 83, 84 corresponding to the sensor unit 3 of the medical treatment system 100 are added, and the mark portion 30 is changed to a plate portion 30A. In FIG. 9, the same components as those in FIG. 2 are denoted by the same reference signs, and descriptions thereof will be omitted.

The holding member 20 of the medical treatment equipment 200 is configured to be wrapped around a trunk portion H of a user. The holding member 20 holds: the electrode pads 61 to 65 and the electrode pads 71 to 75; and the blood flow sensors 81 to 84 and maintains the electrode pads 61 to 65, the electrode pads 71 to 75, and the blood flow sensors 81 to 84 pressed against the body surface of the user.

The holding member 20 includes the plate portion 30A as a positioning portion. The plate portion 30A is disposed substantially in the center of the holding member 20 in the length direction. The plate portion 30A extends along the width direction of the holding member 20. The plate portion 30A is formed of, for example, a resin material having flexibility.

The plate portion 30A is disposed on the first main surface 20a of the holding member 20. Note that the plate portion 30A may be disposed on the second main surface 20b. The electrode pads 61 to 65 and the electrode pads 71 to 75 are disposed at positions substantially line-symmetric with respect to the plate portion 30A.

At the time of wrapping the medical treatment equipment 200 around the trunk portion H, the holding member 20 is wrapped such that the plate portion 30A overlaps with the spine of the user. In this case, an upper end of the holding member 20 is preferably aligned with the navel of the user. The spine and navel of the user are reference positions. The portion in which the plate portion 30A is disposed has high rigidity compared with another portion of the holding member 20 in which the plate portion 30A is not disposed. Therefore, the user can intuitively perceive with a feeling of contact of the highly rigid portion with the back that the plate portion 30A is overlapped with the spine. The plate portion 30A is positioned so as to overlap with the spine, and thus the electrode pads 61 to 65 and the electrode pads 71 to 75 are respectively guided to target positions.

FIG. 9 illustrates a straight line L1 passing through the center position of the plate portion 30A in the length direction of the holding member 20 and extending in the width direction of the holding member 20. Further, FIG. 9 illustrates a straight line L2 passing through the center position in the width direction of the holding member 20 and extending in the length direction of the holding member 20. Furthermore, FIG. 9 illustrates regions A1 to A4 as four regions obtained by dividing the area of the holding member 20 disposed on the back of the user with the straight line L1 and the straight line L2.

The electrode pad 61 and the electrode pad 62 are disposed in the region A1. The electrode pad 71 and the electrode pad 72 are disposed in the region A2. The electrode pad 64 and the electrode pad 65 are disposed in the region A3. The electrode pad 74 and the electrode pad 75 are disposed in the region A4. The electrode pad 63 is disposed across the region A1 and the region A3 at the boundary of the region A1 and the region A3. The electrode pad 73 is disposed across the region A2 and the region A4 at the boundary of the region A2 and the region A4.

The blood flow sensors 81 to 84, in each of which a photoelectric sensor is used for example, are configured to irradiate the body surface of the user with light. The blood flow sensor 81 is disposed between the electrode pads 61, 62 and the electrode pad 63 in the region A1. The blood flow sensor 82 is disposed between the electrode pads 71, 72 and the electrode pad 73 in the region A2. The blood flow sensor 83 is disposed between the electrode pads 64, 65 and the electrode pad 63 in the region A3. The blood flow sensor 84 is disposed between the electrode pads 74, 75 and the electrode pad 73 in the region A4. Measurement information indicating the blood flow measured by the blood flow sensors 81 to 84 is transmitted to the control unit 50 of the main body portion 10.

The blood flow sensor 81 is disposed in the region A1. Therefore, with the medical treatment equipment 200 wrapped around the trunk portion, a portion of the body, which is in contact with the region A1 forms a site to which the blood flow sensor 81 is attached. In addition, the site to which the blood flow sensor 81 is attached is managed in association with a group of the electrode pad 61, the electrode pad 62, and the electrode pad 63.

The blood flow sensor 82 is disposed in the region A2. Therefore, with the medical treatment equipment 200 wrapped around the trunk portion, a portion of the body, which is in contact with the region A2 forms a site to which the blood flow sensor 82 is attached. In addition, the site to which the blood flow sensor 82 is attached is managed in association with a group of the electrode pads 71, the electrode pad 72, and the electrode pad 73.

The blood flow sensor 83 is disposed in the region A3. Therefore, with the medical treatment equipment 200 wrapped around the trunk portion, a portion of the body, which is in contact with the region A3 forms a site to which the blood flow sensor 83 is attached. In addition, the site to which the blood flow sensor 83 is attached is managed in association with a group of the electrode pad 64, the electrode pad 65, and the electrode pad 63.

The blood flow sensor 84 is disposed in the region A4. Therefore, with the medical treatment equipment 200 wrapped around the trunk portion, a portion of the body, which is in contact with the region A4 forms a site to which the blood flow sensor 84 is attached. In addition, the site to which the blood flow sensor 84 is attached is managed in association with a group of the electrode pad 74, the electrode pad 75, and the electrode pad 73.

The function block of the control unit 50 of the main body portion 10 is the same as that of FIG. 6; however, the function of the control unit 50 is different. The measurement information acquisition unit 51A of the medical treatment equipment 200 acquires measurement information from the blood flow sensors 81 to 84. The body state evaluation unit 51B of the medical treatment equipment 200 evaluates a blood circulation state for each of the sections in which the blood flow sensors 81 to 84 are attached, based on the measurement information of each of the blood flow sensors 81 to 84 acquired by the measurement information acquisition unit 51A.

The body state evaluation unit 51B of the medical treatment equipment 200 specifically determines whether the blood flow based on the measurement information is at a predetermined blood flow threshold or higher, for each measurement information acquired from each blood flow sensor 81 to 84. When the blood flow is at the blood flow threshold or higher, a relatively high value (for example, “2”) is set for an evaluation value of a blood circulation state of the site to which the blood flow sensor as the output source of the measurement information is attached. When the blood flow is below the blood flow threshold, a relatively low value (for example, “1”) is set for an evaluation value of a blood circulation state of the site. The evaluation value is not limited to two levels, and may be divided into three or more levels.

The operating state control unit 51C of the medical treatment equipment 200 increases the amount of energy (current amplitude value) generated from the electrode pad corresponding to a site where the evaluation value of the blood circulation state evaluated by the body state evaluation unit 51B is at the threshold TH2 or lower, to be greater than the amount of energy generated from the electrode pad corresponding to a site where the evaluation value of the blood circulation state exceeds the threshold TH2.

Operation of Medical Treatment Equipment 200 During Treatment

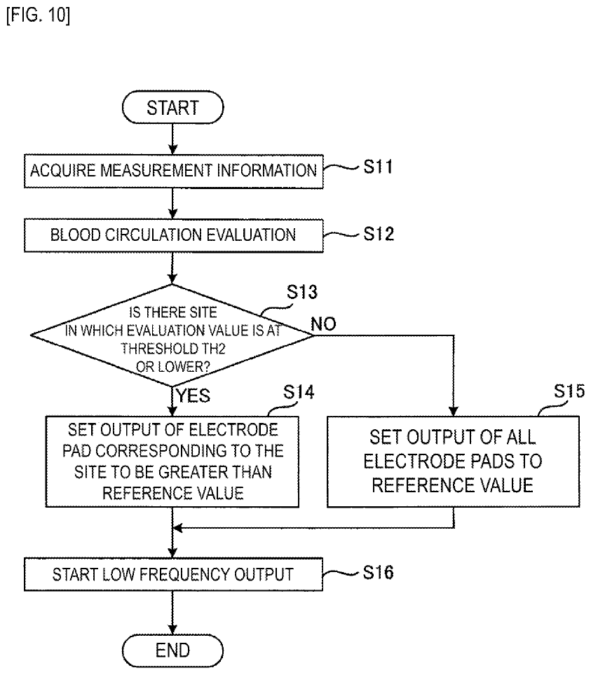

FIG. 10 is a flowchart for illustrating the operation of the medical treatment equipment 200 illustrated in FIG. 9 during treatment. When an instruction to start a low frequency treatment is made by operation of the button 14, the blood flow of the blood flow sensors 81 to 84 is measured by control of the control unit 50, and the measurement information is acquired by the measurement information acquisition unit 51A (step S11).

Next, the body state evaluation unit 51B evaluates, based on the acquired measurement information, a blood circulation state for each of the sites to which the blood flow sensors 81 to 84 are attached (step S12).

Next, the operating state control unit 51C determines whether there is a site where an evaluation value of a blood circulation state is at the threshold TH2 (for example, “1”) or lower (step S13). When there is a site where the evaluation value of the blood circulation state is at the threshold TH2 or lower (step S13: YES), the operating state control unit 51C sets the output (an amplitude value) of a low frequency current supplied to a group of the electrode pads corresponding to a site where the evaluation value of the blood circulation state exceeds the threshold TH2, to a predetermined reference value, and sets the output of a low frequency current supplied to a group of the electrode pads corresponding to a site where the evaluation value of the blood circulation state is at the threshold TH2 or lower, to a value larger than the reference value (step S14).

When there is no site where the evaluation value of the blood circulation state is at the threshold TH2 or lower (step S13: NO), the operating state control unit 51C sets the output of a low frequency current supplied to the electrode pads 61 to 65 and the electrode pads 71 to 75 to the aforementioned reference value (step S15).

After step S14 or step S15, the low frequency current of the amplitude value set in step S14 or step S15 is supplied to the electrode pads 61 to 65 and the electrode pads 71 to 75 (step S16), and thus treatment of the waist region of the user is started.

Effect of Medical Treatment Equipment 200

As described above, according to the medical treatment equipment 200, in a case where, for example, a location in which a blood circulation state is poor and a location in which a blood circulation state is good are mixed in the affected area, a strong low frequency current can be applied to the location in which the blood circulation state is poor. Therefore, improvement in the blood circulation state can be expected, which can lead to symptom improvement of the affected area.

Modified Example of Embodiments

A low-frequency treatment device is taken as an example of the medical treatment equipment 1; however, as described above, the medical treatment equipment 1 may be an ultrasonic treatment device or an electromagnetic wave treatment device. For example, the medical treatment equipment 1 may be configured such that the holding member 20 is replaced by an ultrasonic probe connected to the main body portion 10 and such that the control unit 50 controls ultrasonic output from the ultrasonic probe based on the evaluation results of a state of the body of a user. Also, the medical treatment equipment 1 may be configured such that the holding member 20 is replaced by an electromagnetic wave irradiation device and such that the control unit 50 controls electromagnetic wave output from the electromagnetic wave irradiation device based on the evaluation results of a state of the body of the user.

Further, the medical treatment equipment 1 controls low frequency output based on the evaluation results. Alternatively, for example, when internally including a mechanism that can move electrode pads, the medical treatment equipment can control the arrangement position of the electrode pads based on the evaluation results. Furthermore, when including four groups as electrode pads as in the medical treatment equipment 200, the medical treatment equipment 1 may control which group of the electrode pads to be operated based on the evaluation results. For example, only a group corresponding to a portion with a low evaluation value may be operated, and a group corresponding to a portion with a high evaluation value may be controlled to a stopped state without operation.

While various embodiments have been described with reference to the drawings, needless to say, the present invention is not limited to such examples. It will be apparent to those skilled in the art that various changes and modifications can be made within the scope of the claims, and it is understood that these are naturally belong within the technical scope of the present invention. Further, each of the components of the above-described embodiments may be combined as desired within a range that does not depart from the spirit of the present invention.

Note that the present application is based on a Japanese Patent Application filed on Mar. 13, 2019 (JP 2019-045928), the content of which is incorporated herein by reference.

### REFERENCE SIGNS LIST

100 Medical treatment system
1 Medical treatment equipment
3 Sensor unit
41 First electrode pad
42 Second electrode pad
50 Control unit
51A Measurement information acquisition unit
51B Body state evaluation unit
51C Operating state control unit

1. Medical treatment equipment for treating a body of a user with energy generated by energization, the medical treatment equipment comprising:
a processor configured to:
acquire, from a blood flow sensor attached to a portion of a surface of the body of the user to measure a blood flow, measurement information of the blood flow;
evaluate a blood circulation state of the portion of the body based on the measurement information; and
control an operating state of a generation source of the energy, based on an evaluation result.

2. The medical treatment equipment according to claim 1, wherein in a case where an evaluation value of the blood circulation state is at a threshold or lower, the processor is further configured to increase energy generated from the generation source of the energy, compared with a case where the evaluation value of the blood circulation state exceeds the threshold.

3. The medical treatment equipment according to claim 1, wherein the processor is further configured to acquire the measurement information of the blood flow from a plurality of the blood flow sensors,
the generation source includes electrode pads corresponding to sites of the body of the user to which the plurality of the blood flow sensors are respectively attached, and
the processor is further configured to control an amount of energy generated from each of the electrode pads, based on an evaluation result of the blood circulation state.

4. The medical treatment equipment according to claim 3, wherein the processor is further configured to:
evaluate a blood circulation state for each of the sites to which the blood flow sensors are respectively attached, based on measurement information of each of the blood flow sensors, and
increase an amount of energy generated from the electrode pad corresponding to the site in which an evaluation value of the blood circulation state is at a threshold or lower, to be greater than an amount of energy generated from the electrode pad corresponding to the site in which an evaluation value of the blood circulation state exceeds the threshold.

5. A control method for medical treatment equipment for treating a body of a user with energy generated by energization, the control method comprising:
a measurement information acquisition step of acquiring, from a blood flow sensor attached to a portion of a surface of the body of the user to measure a blood flow, measurement information of the blood flow;
a body state evaluation step of evaluating a blood circulation state of the portion of the body based on the measurement information; and
an operating state control step of controlling an operating state of a generation source of the energy, based on an evaluation result from the body state evaluation step.

6. A non-transitory storage medium storing a control program for medical treatment equipment for treating a body of a user with energy generated by energization, the non-transitory storage medium storing the control program for the medical treatment equipment, allowing a computer to execute:
a measurement information acquisition step of acquiring, from a blood flow sensor attached to a portion of a surface of the body of the user to measure a blood flow, measurement information of the blood flow;
a body state evaluation step of evaluating a blood circulation state of the portion of the body based on the measurement information; and
an operating state control step of controlling an operating state of a generation source of the energy, based on an evaluation result from the body state evaluation step.
